(19) United States
(12) Patent Application Publication
(10) Pub. No.: US 2025/0195390 A1
(43) Pub. Date: Jun. 19, 2025

(54) EYELASH COATING COMPOSITIONS FOR FALSE EYELASHES

(71) Applicant: L'OREAL, Paris (FR)

(72) Inventors: David CHAN, Oradell, NJ (US); Alison Jeanne SCORESE, Rahway, NJ (US); Allison PERNER, Metuchen, NJ (US)

(21) Appl. No.: 18/540,979

(22) Filed: Dec. 15, 2023

(51) Int. Cl. A61K 8/81; A61K 8/06; A61K 8/64; A61K 8/73; A61Q 1/00; C09J 133/08

(52) U.S. Cl. CPC A61K 8/8152; A61K 8/06; A61K 8/64; A61K 8/731; A61Q 1/00; C09J 133/08; A61K 2800/95

(57) ABSTRACT

The invention relates to eyelash and/or eyebrow coating compositions including water, fibers, and at least one latex film-forming agent having a low glass transition temperature (Tg), which can be applied to eyelashes and/or eyebrows (natural or artificial), and which can be used to attach false (artificial) eyelashes and/or eyebrows to natural eyelashes and/or eyebrows, as well as to associated methods.

### FIELD OF THE INVENTION

The present invention relates to eyelash and/or eyebrow coating compositions comprising water, fibers, and at least one latex film-forming agent having a low glass transition temperature (Tg). The eyelash and/or eyebrow coating compositions of the present invention possess sufficient tack and sufficient quick-drying properties such that, after the compositions are applied to eyelashes and/or eyebrows, false or artificial eyelashes and/or eyebrows can be adhered to the eyelashes and/or eyebrows by, through or using the applied eyelash and/or eyebrow coating compositions of the present invention by placing the false or artificial eyelashes and/or eyebrows on the applied composition and allowing the eyelash and/or eyebrow coating composition to dry. Further, the eyelash and/or eyebrow coating compositions can be applied to false (artificial) eyelashes and/or eyebrows or natural (real) eyelashes and/or eyebrows, if desired, to care for or make up the eyelashes and/or eyebrows and, as such, the eyelash and/or eyebrow coating compositions of the present invention can be multi-purpose.

### DISCUSSION OF THE BACKGROUND

Mascaras typically contain wax which can provide body and volume to the composition. Mascaras can also contain surfactants, particularly when they are in the form of emulsions. However, mascara formulations are typically created to minimize or squelch the full effect of the properties of these ingredients, if present, in an attempt to obtain specific desired properties of the mascara being formulated such as volumization, lengthening, easy removal, etc.

And mascaras can contain fibers as well, typically in low amounts of about 0.1% by weight or less with respect to the total weight of the mascara.

Mascara compositions tend to be of two types, (1) water-resistant, long-wearing mascaras which are anhydrous or (2) removable mascaras (for example, removable with soap and water) which contain water. Generally speaking, water-resistant mascaras are not easily removable, and removable mascaras are not particularly long-wearing.

Regarding application of fake eyelashes to eyelashes, an involved, multistep process is typically used to achieve an acceptable look that blends well with a wearer's natural lashes. The cumbersome process includes a wearer first finding the appropriate style of false eyelashes from myriad of choices, trimming the length of the selection to match their natural eyelash size and/or style, applying adhesive to eyelashes, and then finally attaching and positioning the fake lash onto the eyelid or eyelash.

CA1161330A and U.S. patent application publication 2020/0128897 disclose methods which include separate application of liquid adhesive to eyelashes to moisten them, then application of fiber filaments to the moistened eyelashes, and then applying more liquid adhesive to the eyelashes.

Fake (artificial) eyelashes typically can be applied to natural (real) eyelashes through adhesives (glue) or magnets.

For example, US 2016/0206031 discloses the use of magnetic materials, and US 2019/0261715 discloses magnetic materials in an eyeliner-type composition.

With respect to adhesives or glue, US 2017/0027844, WO 2020/172746, and WO 2019/003454 are examples of references disclosing the use of such compounds for false (artificial) eyelashes.

U.S. Pat. No. 9,320,920 discloses compositions for making up eyelashes, including false (artificial) eyelashes.

There remains a need for improved eyelash and/or eyebrow coating compositions having improved cosmetic properties, which can be useful for attaching fake (artificial) eyelashes and/or eyebrows to natural (real) eyelashes and/or eyebrows.

Accordingly, one aspect of the present invention is a water-containing care and/or makeup and/or treatment composition for eyelashes and/or eyebrows which can be used to adhere or bind false (artificial) eyelashes and/or eyebrows to natural (real) eyelashes and/or eyebrows.

### SUMMARY OF THE INVENTION

The present invention relates to eyelash and/or eyebrow coating compositions comprising water, fibers, and at least one latex film-forming agent having a low glass transition temperature (Tg). The eyelash and/or eyebrow coating compositions of the present invention possess sufficient tack and sufficient quick-drying properties such that, after the compositions are applied to eyelashes and/or eyebrows, false or artificial eyelashes and/or eyebrows can be adhered to the eyelashes and/or eyebrows by, through or using the applied eyelash and/or eyebrow coating compositions of the present invention by placing the false or artificial eyelashes and/or eyebrows on the applied composition and allowing the eyelash and/or eyebrow coating composition to dry. Further, the eyelash and/or eyebrow coating compositions can be applied to false (artificial) eyelashes and/or eyebrows or natural (real) eyelashes and/or eyebrows, if desired, to care for or make up the eyelashes and/or eyebrows and, as such, the eyelash and/or eyebrow coating compositions of the present invention can be multi-purpose. Preferably, the composition (1) comprises at least about 0.25% by weight with respect to the total weight of the composition of fibers; and/or (2) further comprises at least one coloring agent, at least one wax, and/or at least one surfactant.

The present invention also relates to methods of caring for and/or making up real (natural) eyelashes and/or eyebrows, or false (artificial) eyelashes and/or eyebrows, by applying compositions of the present invention to natural or fake eyelashes and/or eyebrows in an amount sufficient to care for and/or make up the natural or false eyelashes and/or eyebrows.

The present invention also relates to methods of applying fake (artificial) eyelashes and/or eyebrows to real (natural) eyelashes and/or eyebrows comprising applying compositions of the present invention to natural eyelashes and/or eyebrows, applying fake eyelashes and/or eyebrows to the natural eyelashes and/or eyebrows upon which compositions of the present invention have been previously applied, and allowing the compositions of the present invention to dry so that the fake eyelashes and/or eyebrows adhere or bind to the natural eyelashes and/or eyebrows. Preferably, the composition (1) comprises at least about 0.25% by weight with respect to the total weight of the composition of fibers; and/or (2) further comprises at least one coloring agent, at least one wax, and/or at least one surfactant.

It is to be understood that both the foregoing general description and the following detailed description are exemplary and explanatory only, and are not restrictive of the invention.

### DETAILED DESCRIPTION OF THE INVENTION

In the following description of the invention and the claims appended hereto, it is to be understood that the terms used have their ordinary and accustomed meanings in the art, unless otherwise specified.

“About” as used herein means within 10% of the indicated number (e.g. “about 10%” means 9%-11% and “about 2%” means 1.8%-2.2%).

“A” or “an” as used herein means “at least one.”

“At least one” means one or more and thus includes individual components as well as mixtures/combinations.

As used herein, all ranges provided are meant to include every specific range within, and combination of subranges between, the given ranges. Thus, a range from 1-5, includes specifically 1, 2, 3, 4 and 5, as well as subranges such as and 2-5, 3-5, 2-3, 2-4, 1-4, etc.

“Film former”, “film-forming polymer” or “film-forming agent” or “co-film former” as used herein means a polymer or resin that leaves a film on the substrate to which it is applied, for example, after a solvent accompanying the film former has evaporated, absorbed into and/or dissipated on the substrate.

“Wax” as used herein is a lipophilic fatty compound that is solid at ambient temperature (25° C.) and changes from the solid to the liquid state reversibly, having a melting temperature of more than 30° C. and, for example, more than 45° C., and a hardness of more than 0.5 MPa at ambient temperature.

“Real” and “natural” are used interchangeably throughout this specification.

“False,” “fake” and “artificial” are used interchangeably throughout this specification.

“Surfactant” and “emulsifier” are used interchangeably throughout this specification.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 100° C.

“Non-volatile”, as used herein, means having a flash point of greater than about 100° C. “Polymer” as used herein means a compound which is made up of at least two monomers.

“Polymer” as used herein means a compound which is made up of at least two monomers.

“Free of” or “substantially free of” or “devoid of” as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. Thus, for example, “free of triethanolamine (TEA)” means that an effective amount (that is, more than trace amounts) of TEA is omitted from the composition (that is, about 0% by weight), “substantially free of TEA” means that TEA is are present in amounts less than 1% by weight, and “devoid of TEA” means that TEA is present in amounts less than 0.5% by weight, based on the total weight of the composition. The same nomenclature applies for all other ingredients identified throughout the application and in this paragraph such as, for example, cyanoacrylates (“free of cyanoacrylates,” “substantially free of cyanoacrylates,” and “devoid of cyanoacrylates”) and magnets (“free of magnets,” “substantially free of magnets,” and “devoid of magnets”), even if not specifically discussed for each identified ingredient. As used herein, such phrases should be understood to have meanings consistent with the discussion within this paragraph. As noted, discussed examples of the use of such language are intended to be exemplary, not limiting.

“Tack” as used herein refers to the quality exhibited by compositions that adhere to an object after application to a substrate. Tack may be evaluated by any method known in the art for evaluating it, such as using a texture analyzer. For example, a sample can be applied to a substrate (for example, a 1 mil drawdown), allowed to dry (for example, for 1 minute), and contacted by an object such as an acrylic cylindrical probe (4 cm in height, 1.2 cm diameter base), after which the force associated with removal of the probe from the object can be measured and reported as tack (g). Such measurements can be performed less than 5 minutes after the drying period, such as between 3 and 3½ minutes. Preferably, compositions of the present invention possess tack properties, when determined by this method, of greater than 45 g, preferably greater than 50 g, preferably greater than 70 g, preferably greater than 90 g, and preferably greater than 100 g. So, compositions of the present invention preferably have tack properties ranging from about 30 g to about 200 g, preferably from about 50 g to about 150 g, and preferably from about 70 g to about 130 g, including all ranges and subranges therebetween such as, for example, about 45 g to about 110 g, about 50 g to about 110 g, about 70 g to about 120 g, about 90 g to about 115 g, etc.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to keratin materials such as eyelashes and/or eyebrows and evaluating the color of the composition after an extended period of time. For example, the color of a composition may be evaluated immediately following application to keratin materials such as eyelashes and/or eyebrows and these characteristics may then be re-evaluated and compared after a certain amount of time. Further, these characteristics may be evaluated with respect to other compositions, such as commercially available compositions.

“Making up” as used herein means to provide decoration (for example, color) to the eyelashes and/or eyebrows.

“Protecting” as used herein means to inhibit damage to the eyelashes and/or eyebrows by providing a protective layer on the eyelashes and/or eyebrows such as, for example, through application of the compositions of the present invention and is encompassed within “caring for.”

The compositions and methods of the present invention can comprise, consist of, or consist essentially of the essential elements and limitations of the invention described herein, as well as any additional or optional ingredients, components, or limitations described herein or otherwise useful. For example, the surfactant component of the emulsion composition can “consist essentially of” identified surfactant(s) or types of surfactants discussed below.

For purposes of the present invention, the “basic and novel property” associated with compositions, components and methods which “consist essentially of” identified ingredients or actions is “attaching fake eyelashes and/or eyebrows to natural eyelashes and/or eyebrows.”

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Water

According to present invention, compositions comprising water are provided. The compositions of the present invention are not anhydrous. Preferably, compositions of the present invention comprise from about 20% to about 75% water, preferably from about 25% to about 70% water, preferably from about 30% to about 60% water, and preferably from about 30% to about 55% water by weight with respect to the total weight of the composition, including all ranges and subranges therebetween, such as for example about 30% to about 70%, about 25% to about 60%, about 40% to about 55%, etc. Preferably, the compositions of the present invention are in the form of an emulsion, with a simple emulsion such as an oil-in-water (O/W) or wax-in-water being the most preferred form.

### Fibers

According to the present invention, compositions comprising fibers are provided.

“Fiber” as used herein means a linear object of length L and of diameter D such that L is greater than D, preferably much greater than D, where D is the diameter of the circle in which the cross section of the fiber is inscribed. Preferably, the ratio L/D (or aspect ratio) is from 3.5 to 2500, preferably from 10 to 1000, and preferably from 20 to 500, including all ranges and subranges therebetween.

Suitable fibers include fibers which are synthetic or natural, and mineral or organic, in origin. They may be solid or hollow, curved or straight. They may especially have a circular or polygonal (square, hexagonal or octagonal) cross section. Their ends may be blunted and/or smoothed.

Suitable fibers may be those used in the manufacture of textiles, and especially silk fibers, cotton fibers, wool fibers, flax fibers, cellulose fibers, from vegetables or from algae, rayon fibers, polyamide (Nylon®) fibers, viscose fibers, acetate fibers, especially rayon acetate fibers, acrylic polymer fibers, especially polymethyl methacrylate fibers or poly(2-hydroxyethyl methacrylate) fibers, polyolefin fibers and especially polyethylene or polypropylene fibers, polyethylene terephthalate (PET) fibers, polybutylene terephthalate (PBT) fibers, glass fibers, silica fibers, carbon fibers, especially fibers of carbon in graphite form, polytetrafluoroethylene (such as Teflon®) fibers, insoluble collagen fibers, polyester fibers, polyvinyl chloride fibers or polyvinylidene chloride fibers, polyvinyl alcohol fibers, polyacrylonitrile fibers, chitosan fibers, polyurethane fibers, polyethylene phthalate fibers, or fibers formed from a mixture of polymers such as those mentioned above, for instance polyamide/polyester fibers. Furthermore, the fibers may be optionally surface-treated.

Preferably, the fibers are selected from rayon fibers, silk fibers, and mixtures thereof.

Rayon is typically produced by dissolving cellulose, then converting the dissolved cellulose to insoluble fibrous cellulose. Various processes have been developed for production of rayon, such as, for example, the cuprammonium method, the viscose method, and the lyocell process.

Rayon fibers of varying sizes can be made and are commercially-available. Length of rayon fibers is typically provided in units of millimeters (“mm”). Weight/density of the fibers is typically provided in decitex (Dtex) or denier, where Dtex is the weight in grams per 10,000 meters of fiber, and denier is the weight in grams per 9000 meters of fiber. For example, a dtex value of 3.3 corresponds to a denier value of 3.0.

Rayon fibers are generally commercially available such as, for example, from Daito Kasei Kogyo, Moley Coating Solutions (1.7 Dtex, 0.3-0.6 mm; 3.3 Dtex, 0.3-1 mm), Kobo Products (e.g., Rayon Fiber 3.3T-0.8 MM and Daitosol 5500), Flockit (0.75 mm/3.3dtex) and Claremont Flock.

Generally speaking, fibers of the present invention preferably have a length ranging from 1 μm to 10 mm, preferably from 0.1 mm to 5 mm, preferably from 0.3 mm to 3 mm, preferably from 0.5 mm to 2.0 mm, preferably from about 0.75 mm to about 1.75 mm, preferably from about 0.8 mm to about 1.6 mm, preferably from about 0.9 mm to about 1.5 mm, and preferably from about 1 mm to about 1.5 mm, including all ranges and subranges therebetween such as, for example, about 2 μm to about 50 μm, about 3 μm to about 20 μm, about 3 μm to about 10 μm, about 1 mm to about 1.3 mm, about 0.9 mm to about 1.6 mm, about 1 mm to about 1.5 mm, etc.

The cross section of the fibers may be within a circle of diameter ranging preferably from 2 nm to 500 μm, preferably from 100 nm to 100 μm, and preferably from 1 μm to 50 μm, including all ranges and subranges therebetween.

The weight or yarn count of the fibers may preferably range from 0.1 to 10 denier, preferably from 1 to 7 denier, preferably from about 1.5 denier to about 5 denier and preferably from about 2 denier to about 4 denier, including all ranges and subranges therebetween such as, for example, about 1.5 denier to about 3 denier, about 2 denier to about 3.5 denier, about 4 denier to about 7 denier, etc.

It is to be understood that the present disclosure contemplates fibers having length, cross section and weight characteristics resulting from the combination of properties discussed above, in particular rayon and/or silk fibers. For example, the disclosure contemplates, in particular, rayon and/or silk fibers having a length of about 1 mm to about 1.3 mm and a weight of about 2 denier to about 3.5 denier, with a cross-section as discussed above. Similarly, the disclosure contemplates, in particular, rayon and/or silk fibers having a length of about 0.9 mm to about 1.6 mm and a weight of about 1.5 denier to about 3 denier, with a cross-section as discussed above.

Preferably, the fibers is/are present in the compositions of the present invention in amounts of active material (e.g., solid content) generally ranging from about 0.25% to about 5%, preferably from about 0.3% to about 4%, preferably from about 0.4% to about 3%, and preferably from about 0.5% to about 2%, by weight, based on the total weight of the composition, including all ranges and subranges in between, such as, for example about 0.33% to about 2.25%, about 0.3% to about 5%, about 0.25% to about 2.5%, about 0.4% to about 1.75%, about 0.5% to about 4.3%, etc.

Although not wishing to be bound by any particular theory, it is believed that when fibers are added to compositions of the present invention in combination with latex film-forming agent(s) having a low glass transition temperature (Tg) discussed herein, the fibers and latex(es) in a single composition can increase adhesion between the eyelashes and/or eyebrows and the false or artificial eyelashes and/or eyebrows which are applied onto them (after application of compositions of the present invention to the eyelashes and/or eyebrows). It is believed that such increase in adhesion occurs at least in part by the interaction of the fibers and matrix formed by latex film-forming agent(s) having a low glass transition temperature (Tg), resulting in expanded surface area and additional points of contact for the false or artificial eyelashes to adhere on the eyelashes and/or eyebrows (referred to herein as “tendril effect.). Accordingly, fibers, in particular rayon and/or silk fibers, can be added to compositions of the present invention in an “adhesion increasing effective amount” as just described.

### Latex Film-Forming Agent Having a Low Glass Transition Temperature (Tg)

According to the present invention, compositions

comprising at least one latex film-forming agent having a low glass transition temperature (Tg) are provided.

“Low glass transition temperature” or “Tg” means a glass transition temperature less than −46° C., preferably −47° C. or lower, preferably −50° C. or lower, and preferably −55° C., including all ranges and subranges therebetween such as, for example, −47° C. to −100° C., −47° C. to −75° C., −47° C. to −67° C., −50° C. to −100° C., −50° C. to −75° C., −50° C. to −70° C., etc.

“Latex” is generally obtained by suspension or emulsion polymerization or copolymerization of monomers according to processes that are well known to those of ordinary skill in the art. Such monomers may be chosen in particular from styrene, butadiene, acrylonitrile, chloroprene, vinyl acetate, urethanes, isoprene, isobutylene, and acrylic or methacrylic acid, maleic acid, crotonic acid or itaconic acid or esters or amides thereof.

Suitable latexes include particles having molecular weights of more than 100,000 Da weight average molecular weight, preferably more than 120,000 Da, preferably more than 135,000 Da, and preferably more than 150,000 Da, including all ranges and subranges therebetween.

Suitable polymers for the film-forming particles that may be used in the compositions of the present invention include, but are not limited to, synthetic polymers, free-radical type or polycondensate type polymers, polymers of natural origin, and mixtures thereof.

Preferably, the polymers for the film-forming particles may be selected from vinyl (co)polymers, (meth)acrylic (co)polymers, urethanes (co)polymers, and mixtures thereof. Advantageously, the polymer for the film-forming particles is selected from a styrene-(meth)acrylic and (meth)acrylic copolymer, a vinyl acetate and (meth)acrylic copolymer, and mixtures thereof.

Polymers for the film-forming particles of the free-radical type may be chosen, for example, from vinyl polymers or copolymers, such as acrylic polymers.

Vinyl film-forming polymers can result from the polymerization of monomers comprising at least one ethylenic unsaturation and at least one acidic group and/or esters of these acidic monomers and/or amides of these acidic monomers. Monomers comprising at least one acid group which may be used include, for example, α,β-ethylenic unsaturated carboxylic acids such as acrylic acid, methacrylic acid, crotonic acid, maleic acid or itaconic acid. (Meth)acrylic acid and crotonic acid are, for example, used. Preferably, (meth)acrylic acid is used.

The esters of acidic monomers can be chosen, for example, from (meth)acrylic acid esters (also known as (meth)acrylates), such as (meth)acrylates of an alkyl, for example, a C1-C30 alkyl, such as a C1-C20 alkyl, (meth)acrylates of an aryl, such as a C6-C10 aryl, and (meth)acrylates of a hydroxyalkyl, such as a C2-C6 hydroxyalkyl. Among the alkyl (meth)acrylates that may be mentioned, examples include methyl methacrylate, ethyl methacrylate, butyl methacrylate, isobutyl methacrylate, 2-ethylhexyl methacrylate, lauryl methacrylate and cyclohexyl methacrylate. Among the hydroxyalkyl (meth)acrylates that may be mentioned, examples include hydroxyethyl acrylate, 2-hydroxypropyl acrylate, hydroxyethyl methacrylate and 2-hydroxypropyl methacrylate. Among the aryl (meth)acrylates that may be mentioned, examples include benzyl acrylate and phenyl acrylate. The (meth)acrylic acid esters that may be used are, for example, alkyl (meth)acrylates.

The alkyl group of the esters may be substituted. For example, the alkyl group of the esters may be either fluorinated or perfluorinated, i.e., some or all of the hydrogen atoms of the alkyl group are substituted with fluorine atoms. Further, examples of amides of the acid monomers that may be mentioned include (meth)acrylamides, such as N-alkyl(meth)acrylamides, for example, of a C₂-C₁₂ alkyl. Among the N-alkyl(meth)acrylamides that may be mentioned, examples include N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. For example, these monomers may be polymerized with acid monomers and/or esters thereof and/or amides thereof, such as those mentioned above. Examples of vinyl esters that may be mentioned include vinyl acetate, ethylene vinyl acetate, vinyl neodecanoate, vinyl pivalate, vinyl benzoate and vinyl t-butylbenzoate. Styrene monomers that may be mentioned include styrene and α-methylstyrene.

Among the film-forming polycondensates that may be mentioned, examples include polyurethanes, polyesters, polyesteramides, polyamides, epoxyester resins and polyureas, and modifications or derivatives of any of these.

The polyurethanes may be chosen from anionic, cationic, nonionic or amphoteric polyurethanes, polyurethane-acrylics, polyurethane-polyvinylpyrrolidones, polyester-polyurethanes, polyether-polyurethanes, polyureas and polyurea-polyurethanes, and mixtures thereof.

The polyesters may be obtained, in a known manner, by polycondensation of dicarboxylic acids with polyols, such as diols.

The dicarboxylic acid may be aliphatic, alicyclic or aromatic. Examples of such acids that may be mentioned include: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid and 2,6-naphthalenedicarboxylic acid. These dicarboxylic acid monomers may be used alone or as a combination of at least two dicarboxylic acid monomers. Among these monomers, phthalic acid, isophthalic acid and terephthalic acid may, for example, be used.

The diol may be chosen from aliphatic, alicyclic and aromatic diols. The diol used is, for example, chosen from ethylene glycol, diethylene glycol, triethylene glycol, 1,3-propanediol, cyclohexanedimethanol and 4-butanediol. Other polyols that may be used include glycerol, pentaerythritol, sorbitol and trimethylolpropane.

The polyesteramides may be obtained in a manner analogous to that of the polyesters, by polycondensation of diacids with diamines or amino alcohols. Diamines that may be used include, for example, ethylenediamine, hexamethylenediamine and meta- or para-phenylenediamine. An amino alcohol that may be used is, for example, monoethanolamine.

The polyester may also comprise at least one monomer bearing at least one —SO₃M group, wherein M is chosen from a hydrogen atom, an ammonium ion NH₄⁺ and a metal ion such as an Na⁺, Li⁺, K⁺, Mg²⁺, Ca²⁺, Cu²⁺, Fe²⁺ or Fe³⁺ ion. A difunctional aromatic monomer comprising such an —SO₃M group may, for example, be used.

The aromatic nucleus of the difunctional aromatic monomer also comprising an —SO₃M group as described above may be chosen, for example, from benzene, naphthalene, anthracene, biphenyl, oxybiphenyl, sulfonylbiphenyl and methylenebiphenyl nuclei. Among the difunctional aromatic monomers also comprising an —SO₃M group, mention may be made, for example, of sulfoisophthalic acid, sulfoterephthalic acid, sulfophthalic acid, 4-sulfonaphthalene-2,7-dicarboxylic acid.

The copolymers used are, for example, those based on isophthalate/sulfoisophthalate, such as copolymers obtained by condensation of diethylene glycol, cyclohexanedimethanol, isophthalic acid and sulfoisophthalic acid.

The polymer for the film forming particles may also be a liposoluble polymer. Examples of the liposoluble polymer that may be mentioned include copolymers of a vinyl ester (wherein the vinyl group is directly linked to the oxygen atom of the ester group and the vinyl ester comprises a radical chosen from saturated, linear or branched hydrocarbon-based radicals of 1 to 19 carbon atoms, linked to the carbonyl of the ester group) and of at least one other monomer, which may be a vinyl ester (different from the vinyl ester already present), an α-olefin (comprising from 8 to 28 carbon atoms), an alkyl vinyl ether (the alkyl group of which comprises from 2 to 18 carbon atoms) or an allylic or methallylic ester (comprising a radical chosen from saturated, linear or branched hydrocarbon-based radicals of 1 to 19 carbon atoms, linked to the carbonyl of the ester group).

These copolymers may be crosslinked using crosslinking agents that may be either of the vinylic type or of the allylic or methallylic type, such as tetraallyloxyethane, divinylbenzene, divinyl octanedioate, divinyl dodecanedioate and divinyl octadecanedioate.

Examples of these copolymers which may be mentioned include the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

Further examples of the liposoluble film-forming polymers include liposoluble copolymers, such as those resulting from the copolymerization of vinyl esters comprising from 9 to 22 carbon atoms or of alkyl acrylates or methacrylates, wherein the alkyl radicals comprise from 10 to 20 carbon atoms. Such liposoluble copolymers may be chosen, for example, from polyvinyl stearate, polyvinyl stearate crosslinked with the aid of divinylbenzene, of diallyl ether or of diallyl phthalate copolymers, polystearyl (meth)acrylate, polyvinyl laurate and polylauryl (meth)acrylate copolymers, it being possible for these poly(meth)acrylates to be crosslinked with the aid of ethylene glycol dimethacrylate or tetraethylene glycol dimethacrylate. The liposoluble copolymers described above are known and are described, for example, in French patent application FR-A-2 232 303; they may have a weight-average molecular weight ranging, for example, from 2,000 to 500,000 such as from 4,000 to 200,000.

Among the liposoluble film-forming polymers which may be used herein, mention may also be made, for example, of polyalkylenes such as copolymers of C₂-C₂₀ alkenes, such as polybutene, alkylcelluloses with a linear or branched, saturated or unsaturated C₁-C₈ alkyl radical, for instance ethylcellulose and propylcellulose, homopolymers or copolymers of vinylpyrrolidone (VP), such as copolymers of vinylpyrrolidone and of C₂-C₄₀ alkene such as C₃-C₂₀ alkene. In addition to PVP homopolymers, among the VP copolymers which may be used herein, mention may be made, for example, of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

Preferred latexes include latexes which either contain or lack styrene groups.

According to certain embodiments, suitable latex lacking styrene groups are copolymers comprising two or more monomers chosen from acrylic acid, methacrylic acid, and their simple esters, for example, lower alkyl esters such as methyl, ethyl, and ethylhexyl esters. For example, copolymers may be chosen from acrylates copolymers, ammonium acrylates copolymers, ethyl acrylates copolymers, acrylates/ethylhexylacrylate copolymers, acrylates/octylacrylates copolymers, alkyl (meth)acrylates copolymers, acrylates/C₁₂-C₂₂ alkylmethacrylate copolymers, ethylacrylate/methacrylic acid copolymer, ethylhexyl acrylate/butyl acrylate/methacrylic acid, methyl methacrylate/butyl acrylate/methacrylic acid and t-butyl acrylate/ethyl acrylate/methacrylic acid copolymer.

Examples of commercially available acrylate copolymers include, but are not limited to, those available from Kobo Products, Inc. and Daito Kasei Kogyo Co., Ltd., under the trade names Daitosol 5500GX and 5500GM (Tg of −65° C.), and from Dow under the tradename Acudyne 5800P (Tg −49° C.), and commercially available polyurethanes include, but are not limited to, those available from Covestro such as polyurethane-34 under the trade name Baycusan C 1001 (Tg −51° C.).

Preferably, the at least one latex is present in the compositions of the present invention in an amount of active material (that is, an amount not including the aqueous liquid phase of the latex) of from about 10% to about 80%, preferably from about 15% to about 75%, preferably from about 25% to about 70%, preferably from about 40% to about 67%, and preferably from about 50% to about 55% by weight, based on the total weight of the composition, including all ranges and subranges in between, such as from about 30% to about 55%, about 35% to about 65%, about 12.2% to about 25%, about 50% to about 60%, etc.

Preferably, the at least one latex film-forming agent having a low glass transition temperature (Tg) (active material) and the fibers are present in a weight ratio of about 5:1 to about 500:1, preferably about 10:1 to about 300:1, preferably about 100:1 to about 250:1, and preferably about 150:1 to about 225:1, including all ranges and subranges therebetween, such as for example about 125:1 to about 300:1, about 140:1 to about 240:1, etc. Preferably, more latex film-forming agent having a low glass transition temperature (Tg) (active material) is present in the compositions of the present invention than fibers.

### Additional Film Forming Agent

According to preferred embodiments of the present invention, compositions optionally further comprising, in addition to the at least one latex film-forming agent having a low glass transition temperature (Tg), one or more additional film-forming agents suitable for use in compositions for application to fake or natural eyelash and/or eyebrows are provided. Such film-forming agents can be, for example, water-soluble or liposoluble. Acceptable film-forming are known in the art and include, but are not limited to, those disclosed in U.S. patent application publication no. 2004/0170586, the entire contents of which is hereby incorporated by reference.

Specific examples of film-forming agents include, but are not limited to, proteins, such as proteins of plant origin, such as, for example, wheat or soya proteins; or proteins of animal origin, such as keratins, for example keratin hydrolysates and sulfonic keratins; cellulose polymers, such as, for example, hydroxyethylcellulose, hydroxypropylcellulose, methylcellulose or ethylhydroxyethylcellulose; gums arabic, guar gum, xanthan derivatives or karaya gum; alginates and carrageenans; glycoaminoglycans, hyaluronic acid and its derivatives; shellac resin, other gums such as, for example, gum acacia and gum sandarac, dammars, elemis or copals; muccopolysaccharides, such as chondroitin sulfates, and mixtures thereof.

Specific examples of suitable polymers further include, but are not limited to, polyalkylenes, polyvinylalcohols (PVA), polyvinylpyrrolidone (PVP) or vinylpyrrolidone (VP) homopolymers or copolymers, copolymers of a C₂ to C₃₀, such as C₃ to C₂₂ alkene, and combinations thereof. As specific examples of VP copolymers which can be used in the invention, mention may be made of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate copolymer, and mixtures thereof.

If present, the at least one additional film-forming polymer(s) is/are present in the compositions of the present invention in amounts of active material (e.g., solid content) generally ranging from about 0.5% to about 20%, preferably from about 1% to about 15%, preferably from about 2% to about 12.5%, and preferably from about 2.5% to about 10%, by weight, based on the total weight of the composition, including all ranges and subranges in between, such as, for example 8% to 18%, 2% to 10%, etc.

### Surfactant Component

According to preferred embodiments of the present invention, compositions optionally further comprising at least one surfactant are provided. Generally speaking, acceptable surfactants (emulsifiers) can be chosen from ionic emulsifiers, nonionic emulsifiers, and mixtures thereof.

“HLB” refers to the “hydrophilic-lipophilic balance” associated with emulsifiers. In particular, “HLB” value relates to the ratio of hydrophilic groups and lipophilic groups in emulsifiers, and also relates to solubility of the emulsifiers. Lower HLB emulsifiers (less than 8 and preferably less than 6) are more soluble in oils (lipophilic material) and are more appropriate for use in water-in-oil (W/O) emulsions. Higher HLB emulsifiers (greater than 8 and preferably greater than 9) are more soluble in water (hydrophilic material) and are more appropriate for oil-in-water (O/W) emulsions.

By way of example, the following emulsifiers have been reported to have the following HLB values:
Propylene Glycol Isostearate HLB=2.5;
Glyceryl Stearate HLB=3.8;
Sorbitan Isostearate HLB=4.7;
Oleth-2 HLB=4.9;
Glyceryl Laurate HLB=5.2;
Ceteth-2 HLB=5.3;
Methyl Glucose Sesquistearate HLB=6.6;
Ceteth-30 HLB=16.5;
C12-13 pareth-23 HLB=16.7;
Polysorbate 20 HLB=16.7;
Laureth-23 HLB=16.9;
PEG-100 Stearate HLB=18.8; and
Sodium lauryl sulfate HLB=40.

According to preferred embodiments, compositions of the present invention comprise at least one surfactant which has an HLB value which is greater than 8, and at least one surfactant which has an HLB value of less than 8.

According to preferred embodiments, one or more of the emulsifiers is a fatty alcohol, a fatty acid, or ester thereof, optionally alkoxylated (ethoxylated, propoxylated, etc.), glycerylated and/or pegylated. Fatty acids correspond the formula R—COOH and fatty alcohols correspond to the formula R—OH, in which R denotes a saturated or unsaturated hydrocarbon radical preferably having from 7 to 45 carbon atoms, preferably from 9 to 35 carbon atoms, preferably from 15 to 35 carbon atoms, preferably from 15 to 21 carbon atoms, and preferably from 16 to 18 carbon atoms. Mention may be made of, for example, lauric acid/alcohol, stearic acid/alcohol, oleic acid/alcohol, behenyl acid/alcohol, cetyl acid/alcohol and mixtures thereof (including cetearyl compounds).

Suitable emulsifiers include ethoxylated fatty acids or alcohols, ethoxylated fatty acids, partial glycerides of ethoxylated fatty acids or alcohols, glycerolated fatty acids or alcohols, and mixtures thereof.

Suitable alkoxylated fatty alcohols include, for example, the addition products of ethylene oxide with lauryl alcohol, in particular those containing from 2 to 250 oxyethylenated groups (having CTFA names Laureth-2 to Laureth-250); the addition products of ethylene oxide with behenyl alcohol, in particular those containing from 2 to 250 oxyethylenated groups (having CTFA names Beheneth-2 to Beheneth-250); the addition products of ethylene oxide with cetearyl alcohol (mixture of cetyl alcohol and of stearyl alcohol) in particular those containing from 2 to 250 oxyethylenated groups (having CTFA names Ceteareth-2 to Ceteareth-250 such as, for example, Ceteareth-33); the addition products of ethylene oxide with cetyl alcohol, in particular those containing from 2 to 250 oxyethylenated groups (having CTFA names Ceteth-2 to Ceteth-250 such as, for example, Ceteth-30); the addition products of ethylene oxide with stearyl alcohol, in particular those containing from 2 to 250 oxyethylenated groups (having CTFA names Steareth-2 to Steareth-250 such as, for example, and Steareth-20); the addition products of ethylene oxide with isostearyl alcohol, in particular those containing from 2 to 250 oxyethylenated groups (having CTFA names Isosteareth-2 to Isosteareth-250); and mixtures thereof, wherein the amount of alkoxylation preferably ranges from 2 to 250, and preferably from 5 to 200, including all ranges and subranges therebetween including, for example, 10 to 100, 50 to 150, etc.

Suitable alkoxylated fatty acid include, for example, the addition products of ethylene oxide with lauric acid, palmitic acid, stearic acid or behenic acid, and mixtures thereof, in particular those containing from 2 to 250 oxyethylenated groups, such as, for example, laurates of PEG-2 to PEG-50 (having CTFA names: PEG-2 laurate to PEG-50 laurate); palmitates of PEG-2 to PEG-50 (having CTFA names: PEG-2 palmitate to PEG-50 palmitate); stearates of PEG-2 to PEG-250 (having CTFA names: PEG-2 stearate to PEG-250 stearate such as PEG-100 stearate and PEG-200 stearate); palmitostearates of PEG-2 to PEG-50; behenates of PEG-2 to PEG-50 (having CTFA names: PEG-2 behenate to PEG-50 behenate); and mixtures thereof, wherein the amount of alkoxylation preferably ranges from 2 to 250, and preferably from 5 to 200, including all ranges and subranges therebetween including, for example, 10 to 100, 50 to 150, etc.

Suitable glycerylated fatty acids include, for example, glyceryl stearate, glyceryl oleate and glyceryl caprylate.

According to preferred embodiments, the surfactant component may additionally contain or comprise at least one alkyl phosphate surfactant as disclosed, for example, in U.S. Pat. No. 9,687,426, the entire contents of which is hereby incorporated by reference. Preferably, if present, the alkyl phosphate surfactant is chosen from C14-C24, preferably C16-C18 alkyl phosphates, and mixtures thereof. Even more preferably, they are chosen from cetyl phosphate, stearyl phosphate and cetearyl phosphate. For example, cetyl phosphate is commercially available under the names Amphisol K (Roche), Amphisol A (Roche), Arlatone MAP (Uniqema) and Crodafos MCA (Croda). It is to be understood that “alkyl phosphate” includes salts of such compounds such as potassium cetyl phosphate.

Preferred surfactants include at least one selected from the group of palmitic acid, potassium cetyl phosphate, ceteareth-33, steareth-2, steareth-20, glyceryl stearate and stearic acid.

If present, the surfactant(s) (surfactant component) is/are preferably present in the compositions of the present invention in an amount of from about 1% to about 20%, preferably from about 5% to about 17.5%, preferably from about 5% to about 15%, and preferably from about 6% to about 12%, by weight based on the total weight of the composition, including all ranges and subranges in between.

According to some preferred embodiments, however, compositions of the present invention are free of, substantially free of, or devoid of surfactants as defined above.

### Structuring Agents

According to preferred embodiments of the present

invention, compositions optionally further comprising at least one structuring agent are provided. Suitable structuring agents include, but are not limited to, fatty acids or alcohols and waxes.

Suitable fatty acids or alcohols include compounds containing 8 or more carbon atoms which are solid at or above ambient temperature such as, for example, solid at 30° C., preferably solid at 35° C., preferably solid at 40° C., and preferably solid at 45° C. Examples of such compounds include stearic acid, cetyl alcohol, stearyl alcohol, cetearyl alcohol, etc.

Suitable waxes can be hydrocarbon, fluorinated and/or silicone, and be of plant, mineral, animal and/or synthetic origin.

Suitable examples of waxes that can be used in accordance with the present disclosure include those generally used in the cosmetics field: they include those of natural origin, such as beeswax, carnauba wax, candelilla wax, ouricoury wax, Japan wax, cork fibre wax or sugar cane wax, rice or rice bran wax, montan wax, paraffin wax, lignite wax or microcrystalline wax, ceresin or ozokerite, and hydrogenated oils such as hydrogenated castor oil or jojoba oil; synthetic waxes such as the polyethylene waxes obtained from the polymerization or copolymerization of ethylene, and Fischer-Tropsch waxes, or else esters of fatty acids, such as octacosanyl stearate, glycerides which are concrete at 30° C., for example at 45° C., silicone waxes, such as alkyl- or alkoxydimethicones having an alkyl or alkoxy chain ranging from 10 to 45 carbon atoms, poly(di)methylsiloxane esters which are solid at 30° C., and whose ester chain comprising at least 10 carbon atoms, or else di(1,1,1-trimethylolpropane) tetrastearate, which is sold or manufactured by Heterene under the name HEST 2T-4S, and mixtures thereof.

Further suitable examples of wax include, but are not limited to, BIS-PEG-12 DIMETHICONE CANDELILLATE wax such as for example the Siliconyl Candelilla Wax marketed by the company KOSTER KEUNEN, hydrogenated Jojoba wax such as for example that marketed by the company DESERT WHALE, hydrogenated palm oil such as that marketed by the company SIO, rice bran wax, Sumac wax, ceresin waxes, laurel wax, Chinese insect wax, Shellac wax, hydrogenated olive oil such as Waxolive from the company SOLIANCE, waxes obtained by hydrogenation of olive oil esterified with C12 to C18 chain fatty alcohols such as those sold by the company SOPHIM under the brand names Phytowax Olive 12L44, 14L48, 16L55 and 18L57, waxes obtained by hydrogenation of castor oil esterified with cetyl or behenyl alcohol such as for example those which are sold under the names Phytowax Ricin 16 L 64 and Phytowax Ricin 22 L 73 by the company SOPHIM, hydrogenated Cameline wax, Ouricury wax, Montan wax, ozokerite waxes such as for example Wax SP 1020 P marketed by the company Strahl & Pitsch, microcrystalline waxes such as for example that sold under the brand name Microwax HW by the company PARAMELT, triglycerides of lauric, palmitic, cetylic and stearic acids (INCI name: hydrogenated coco glycerides) such as for example that sold under the brand name Softisan 100 by the company SASOL, polymethylene waxes such as for example that sold under the brand name Cirebelle 303 by the company SASOL, polyethylene waxes such as for example those sold under the brand names Performalene 400 polyethylene, Performalene 655 polyethylene and Performalene 500-L polyethylene by the company New Phase Technologies, alcohol-polyethylene waxes such as for example that marketed under the name Performacol 425 Alcohol by the company BARECO, the 95/5 ethylene/acrylic acid copolymer sold under the brand name AC 540 wax by the company Honeywell, hydroxyoctacosanyl hydroxy-stearate such as for example that sold under the brand name Elfacos C 26 by the company AKZO, octacosanyl stearate such as for example that marketed under the name Kester Wax K 82H by the company KOSTER KEUNEN, stearyl stearate such as for example that marketed under the name Liponate SS by the company LIPO CHEMICALS, pentaerythritol distearate such as for example that marketed under the name Cutina PES by the company COGNIS, the mixture of dibehenyl adipate, dioctadecyl adipate and di-eicosanyl adipate (INCI name C18-C22 dialkyl adipate), the mixture of dilauryl adipate and ditetradecyl adipate (INCI name: C12-C14 dialkyl adipate), the mixture of dioctadecyl sebacate, didocosyl sebacate and dieicosyl sebacate (INCI name: C18-C22 dialkyl sebacate) and the mixture of dioctadecyl octadecanedioate, didocosyl octanedioate and dieicosyl octanedioate (INCI name: C18-C22 dialkyl octanedioate) such as for example those marketed by the company COGNIS, pentaerythrityl tetrastearate such as for example Liponate PS-4 from the company Lipo Chemicals, tetracontanyl stearate such as for example Kester Wax K76H from the company KOSTER KEUNEN, stearyl benzoate such as for example Finsolv 116 from the company FINETEX, behenyl fumarate such as for example Marrix 222 from the company AKZO BERNEL, di-(trimethylol-1,1,1-propane) tetrastearate such as for example that which is offered under the name “HEST 2T-4S” by the company HETERENE, didotriacontanyl distearate such as for example Kester Wax K82D from the company KOSTER KEUNEN, polyethylene glycol montanate with 4 ethylene oxide units (PEG-4) such as for example that which is sold under the brand name Clariant Licowax KST1, hexanediol disalicylate such as for example Betawax RX-13750 marketed by the company CP Hall, dipentaerythritol hexastearate such as for example that which is sold under the brand name Hest 2P-6S by the company HETERENE, ditrimethylolpropane tetrabehenate such as for example that which is sold under the brand name Hest 2T-4B by the company HETERENE, Jojoba esters such as for example that which is sold under the brand name Floraester HIP by the company FLORATECH, mixtures of linear (C20-40) carboxylic acid/saturated hydrocarbons (INCI name: C20-40 acid polyethylene) such as for example Performacid 350 acid from the company NEW PHASE TECHNOLOGIES, synthetic wax of the Fischer-Tropsch type such as that marketed under the name Rosswax 100 by the company ROSS, cetyl alcohol, stearyl alcohol, behenyl alcohol, dioctadecyl carbonate such as for example Cutina KE 3737, saccharose polybehenate such as for example Crodaderm B from the company CRODA, waxes of plant origin such as carnauba wax, candelilla wax, hydrogenated jojoba wax, sumac wax, waxes obtained by hydrogenation of olive oil esterified with C12 to C18 chain fatty alcohols sold by the company SOPHIM in the Phytowax range (12L44, 14L48, 16L55 and 18L57), rice bran wax, cetyl, laurel wax, Ouricury wax and mixtures thereof can be mentioned.

Preferred structuring agents include at least one selected from the group of cetearyl alcohol, carnauba wax, glyceryl behenate, glyceryl dibehenate, tribehenin, bis-diglyceryl polyacyladipate-2, paraffin, cetyl alcohol, hydrogenated jojoba oil, hydrogenated palm oil, oryza sativa (rice) bran wax, jojoba butter, synthetic beeswax and candilla wax.

If present, the structuring agent(s) is/are preferably present in the compositions of the present invention in a total amount ranging from 5% to about 30% by weight, preferably from about 7.5% to about 25% by weight, and preferably from about 10% to about 20% by weight, based on the total weight of the composition, including all ranges and subranges within these ranges such as, for example, 8% to 17%, 10% to 25%, 20% to 30%, etc.

According to some preferred embodiments, however, compositions of the present invention are free of, substantially free of, or devoid of structuring agents as defined above.

According to some preferred embodiments, however, compositions of the present invention are free of, substantially free of, or devoid of waxes as defined above.

### Oil Phase

According to embodiments of the present invention, the compositions of the present invention may optionally further comprise at least one oil. “Oil” means any non-aqueous medium which is liquid at ambient temperature (25° C.) and atmospheric pressure (760 mm Hg). Suitable oils can be volatile or non-volatile.

Suitable oils include volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Suitable oils include non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, and for example, the oils sold under the trade names of Isopar or Permethyl. Preferably, the volatile non-silicone oils have a flash point of at least 40° C.

Suitable oils include synthetic oils or esters of formula R₅COOR₆ in which R₅ represents a linear or branched higher fatty acid residue containing from 1 to 40 carbon atoms, including from 7 to 19 carbon atoms, and R6 represents a branched hydrocarbon-based chain containing from 1 to 40 carbon atoms, including from 3 to 20 carbon atoms, with R₆+R7≥10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; pentaerythritol esters; and synthetic ethers containing from 10 to 40 carbon atoms.

If present, the oil(s) is/are preferably present in the compositions of the present invention in an amount ranging from about 0.1% to about 20% by weight, more preferably from about 0.4% to about 15% by weight, and preferably from about 0.5% to about 10% by weight, based on the total weight of the composition, including all ranges and subranges within these ranges.

According to preferred embodiments, however, compositions of the present invention are free of, substantially free of, or devoid of oils as defined above.

According to preferred embodiments, however, compositions of the present invention are substantially free of, devoid of, or free of volatile oils such as, for example, isododecane and/or cyclomethicone.

According to preferred embodiments, compositions of the present invention are substantially free of, devoid of, or free of silicone oils such as, for example, dimethicone and/or cyclomehticone.

According to preferred embodiments, the oil component of the compositions of the present invention consists of non-volatile oils.

According to preferred embodiments, the oil component of the compositions of the present invention consists of hydrocarbon oils.

According to preferred embodiments, the oil component of the compositions of the present invention consists of non-volatile hydrocarbon oils.

### Coloring Agents

According to preferred embodiments of the present

invention, compositions optionally further comprising at least one coloring agent are provided. Preferably, such colored compositions can be cosmetic compositions such as mascaras.

According to this embodiment, the at least one coloring agent is preferably chosen from pigments, dyes, such as liposoluble dyes, nacreous pigments, and pearling agents.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%, including all ranges and subranges therebetween.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, may be present in the composition in a concentration ranging up to 50% by weight of the total weight of the composition, such as from 0.1% to 20%, preferably from 0.1% to 15%, including all ranges and subranges therebetween.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the at least one coloring agent may be present in the composition in a concentration ranging up to 25% by weight of the total weight of the composition, such as from 2.5% to 20%, and further such as from 5% to 15%, including all ranges and subranges therebetween.

According to some preferred embodiments, however, compositions of the present invention are free of, substantially free of, or devoid of coloring agents as defined above.

### Additional Additives

The composition of the invention can also comprise any additive usually used in the field under consideration. For example, dispersants such as poly(12-hydroxystearic acid), antioxidants, essential oils, sunscreens, preserving agents, fragrances, fillers, neutralizing agents, cosmetic and dermatological active agents such as, for example, emollients, moisturizers, vitamins, essential fatty acids, silicone elastomers, pasty compounds, and mixtures thereof can be added. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9th ed. 2002).

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the composition of the invention should be cosmetically or dermatologically acceptable, i.e., it should contain a non-toxic physiologically acceptable medium and should be able to be applied to the eyelash and/or eyebrows of human beings.

According to preferred embodiments of the present invention, methods of caring for and/or making up eyelashes and/or eyebrows (artificial or natural) by applying compositions of the present invention to the eyelashes and/or eyebrows in an amount sufficient to care for and/or make up the eyelashes and/or eyebrows are provided. According to some embodiments, “making up” the keratin material includes applying a composition comprising at least one coloring agent to the eyelashes and/or eyebrows in an amount sufficient to provide color to the eyelashes and/or eyebrows.

In accordance with the preceding preferred embodiments, the compositions of the present invention are applied topically to the desired area of the eyelashes and/or eyebrows in an amount sufficient to care for and/or make up the eyelashes and/or eyebrows. The compositions may be applied to the desired area as needed, preferably once or twice daily, more preferably once daily and then preferably allowed to dry before subjecting to contact such as with clothing or other objects (for example, a topcoat). Preferably, the composition is allowed to dry for about 1 minute or less, more preferably for about 45 seconds or less. The composition is preferably applied to the desired area that is dry or has been dried prior to application, or to which a basecoat has been previously applied.

According to preferred embodiments of the present invention, methods of applying fake (artificial) eyelashes and/or eyebrows to real (natural) eyelashes and/or eyebrows comprising applying compositions of the present invention to natural eyelashes and/or eyebrows, applying fake eyelashes and/or eyebrows to the natural eyelashes and/or eyebrows upon which compositions of the present invention have been previously applied, and allowing the compositions of the present invention to dry so that the fake eyelashes and/or eyebrows attach to the natural eyelashes and/or eyebrows are provided. Preferably, the composition (1) comprises at least about 0.25% by weight with respect to the total weight of the composition of fibers; and/or (2) further comprises at least one coloring agent, at least one wax, and/or at least one surfactant.

In accordance with the preceding preferred embodiments, the compositions of the present invention are applied topically to the desired area of the eyelashes and/or eyebrows in an amount sufficient to attach false (artificial) eyelashes and/or eyebrows to natural eyelasesh and/or eyebrows. The compositions may be applied to the desired area as needed, preferably once or twice prior to attachment of the artificial eyelashes and/or eyebrows, and then allowed to dry after contacting the applied composition with artificial eyelashes and/or eyebrows for attachment to eyelashes and/or eyebrows. Preferably, the artificial eyelashes and/or eyebrows are contacted with the applied composition less than 1 minute after the composition has been applied to natural eyelashes and/or eyebrows, more preferably less than 45 seconds after application.

Typical removal methods and solutions can be used to remove the artificial eyelash and/or eyebrows from natural eyelash and/or eyebrows, if desired.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example IA—Sample Glue Formulations

| Ingredient | Preferred Range | Specific Range |
|---|---|---|
| Latex with low Tg | 40-67% | 50-55% |
| (active material) | | |
| Water | QS | QS |
| Fiber | 0.25-5% | 0.25-2.5% |
| Other (preservative, filler, | 0-5% | 0-2.5% |
| etc.) | | |

### Example IB—Sample Mascara Formulations

| Ingredient | Preferred Range | Specific Range |
|---|---|---|
| Latex with low Tg | 10-30% | 10-20% |
| (active material) | | |
| Coloring Agent | 3-20% | 5-10% |
| Wax | 3-20% | 5-15% |
| Surfactant | 3-20% | 5-15% |
| Water | QS | QS |
| Fiber | 0.25-5% | 0.25-2.5% |
| Other (preservative, filler, | 0-10% | 0-10% |
| etc.) | | |

### Example II—Composition Preparation

Compositions corresponding to Example 1A can be prepared by combining all ingredients in a known manner, such as by speedmixing using a Hauschild SpeedMixer® at 2000 RPM for 2-5 min.

### Example III—Testing Invention Compositions

Each prepared composition below (invention and comparative) was tested by using a 1 mil drawdown bar to form a film, and repeatedly contacting the film in cycles with a P/0.5 HS probe (5.8 cm in length, with the last 0.5 cm beveled to a hemisphere) during drying. The test speed used for all probe motion (downward to the film, and upward away from the film) was 2 mm/s, with instantaneous contact to the film in each cycle, and approximately a 1.4 s lag in between cycles. For each contact, each film was observed to note any characteristic “tendril effect”; that is, the appearance of a thin filament suspended from the tip of the probe. The duration of “tendril effect” for each inventive and comparative example prepared was recorded, with particular focus on tendril effect reappearance after the film was dry, and duration of the effect.

### Test A—Invention Compositions

| Ingredients | Ex 1 | Ex 2 | Ex 3 | Ex 4 |
|---|---|---|---|---|
| Acrylates/ | 55 | 55 | 55 | 55 |
| Ethylhexyl | | | | |
| acrylate | | | | |
| copolymer | | | | |
| (Tg = −66° C.) | | | | |
| Water | QS | QS | QS | QS |
| Linear Rayon | 0.3 | | | |
| Fibers (1 | | | | |
| mm) | | | | |
| Linear Rayon | | 0.3 | | |
| Fibers (1.3 | | | | |
| mm) | | | | |
| Linear Rayon | | | | 1 |
| Fibers (3 | | | | |
| mm) | | | | |
| Linear Silk | | | 0.3 | |
| Fibers | | | | |
| Appearance | Yes— | Yes— | Yes— | Yes— |
| of tendril | appeared | appeared | appeared | appeared |
| | while drying | while drying | while drying | while drying |
| | (60 s) and | (70 s) and | (45 s) and | (70 s) and |
| | endured after | endured after | endured after | endured after |
| | film fully dry | film fully dry | film fully dry | film fully dry |

The above testing demonstrated that invention compositions resulted in persistent tendril using different fibers of different lengths.

### Test B—Testing Invention and Comparative Compositions

| | Ex 1 | | | |
|---|---|---|---|---|
| | (invention | | | |
| | from | Ex 5 | Ex 6 | C1 |
| Ingredients | above ) | (invention) | (invention) | (comparative) |
| Acrylates/Ethylhexyl | 55 | 55 | 52 | 55 |
| acrylate copolymer | | | | |
| (Tg = −66° C.) | | | | |
| Water | QS | QS | QS | QS |
| Linear Rayon (1 | 0.3 | 1 | 5 | 0.03 |
| mm) | | | | |
| Appearance of | Yes— | Yes— | Yes— | No— |
| tendril | appeared | appeared | appeared | Fleeting, |
| | while | while | while | appeared |
| | drying | drying | drying | only during |
| | (60 s) and | (60 s) and | (80 s) and | drying of film |
| | endured | endured | endured | (70 s) and |
| | after film | after film | after film | disappeared |
| | fully dry | fully dry | fully dry | once film was |
| | | | | dry |

Persistence of the tendril effect was thus demonstrated at concentrations ranging from 0.3% to 5%.

### Test C—Comparative Compositions vs. Example 1

TABLE 3
Comparative examples around filler morphology and
absence of filler

| | Ex 1 | | | |
|---|---|---|---|---|
| | (invention | | | |
| | from | C2 | C3 | C4 |
| Ingredients | above) | (comparative) | (comparative) | (comparative) |
| Acrylates/ | 55 | 55 | 55 | 55 |
| Ethylhexyl | | | | |
| acrylate | | | | |
| copolymer | | | | |
| (Tg = −66° C.) | | | | |
| Water | QS | QS | QS | QS |
| Spherical | | 0.3 | | |
| Nylon-12 | | | | |
| fiber | | | | |
| Spherical | | | 0.3 | |
| cellulose | | | | |
| Linear | 0.3 | | | |
| Rayon (1 | | | | |
| mm) | | | | |
| Appearance | Yes— | No— | No— | No— |
| of tendril | appeared | Fleeting, | Fleeting, | Fleeting, |
| | while | appeared | appeared | appeared |
| | drying | only during | only during | only during |
| | (60 s) and | drying of film | drying of film | drying of film |
| | endured | (70 s) and | (70 s) and | (70 s) and |
| | after film | disappeared | disappeared | disappeared |
| | fully dry | once film was | once film was | once film was |
| | | dry | dry | dry |

Persistence of the tendril effect was thus demonstrated for linear fibers.

### Test D—Comparative Compositions with Polymers having Higher Tg

| | C5 | C6 |
|---|---|---|
| Ingredients | (Trial 12) | (Trial 13) |
| Acrylates | 42.75 | 47.5 |
| copolymer | | |
| Water | QS | QS |
| Rayon (1 mm) | 5 | 5 |
| Tg (° C.) | 13.6 | −14 |
| Appearance of | No | No |
| tendril | | |

Absence of the tendril effect was thus demonstrated for compositions having a higher Tg polymer.

### Test E—Comparative Compositions with Polymers having Higher Tg

TABLE 5
Inventive and comparative examples around Acrylates
copolymers with low Tg

| | Ex 6 | C7 | C8 |
|---|---|---|---|
| Ingredients | (Trial 6) | (Trial 15) | (Trial 16) |
| Acrylates/Ethylhexyl | 52.5 | | |
| Acrylate copolymer | | | |
| Polyurethane-35 | | 39 | |
| Acrylates copolymer | | | 48.4 |
| Water | QS | QS | QS |
| Rayon (1 mm) | 5 | 5 | 5 |
| Tg (° C.) | −66 | −46 | −49 |
| Appearance of | Yes—appearing | No | Yes— |
| tendril | while drying (80 s) | | appearing |
| | and enduring after | | only after |
| | film fully dry | | film fully dry |
| | | | (120 s) and |
| | | | enduring |
| | | | thereafter |

Observations of the compositions above demonstrated the efficacy of the low Tg film forming agent when combined with the fibers disclosed above with respect to producing a tendril effect. This tendril effect, which could be conceptualized as a fiber-latex matrix interaction, unexpectedly helps facilitate attachment of artificial eye lashes and/or eyebrows through increased points of contact.

What is claimed is:

1. An eyelash and/or eyebrow coating composition comprising water, from about 0.25% to about 5% of fibers by weight with respect to the total weight of the composition, and at least one latex film-forming agent having a low glass transition temperature (Tg).

2. The composition of claim 1, wherein the composition comprises the at least one fiber in an amount of from about 0.25% to about 4% by weight with respect to the total weight of the composition, preferably from about 0.25% to about 2.5% by weight with respect to the total weight of the composition.

3. The composition of claim 1, wherein the fibers are selected from rayon fibers, silk fibers and mixtures thereof.

4. The composition of claim 1, wherein the fibers are rayon fibers, preferably having a length of about 1 mm to about 1.3 mm.

5. The composition of claim 4, wherein the fibers have a weight of about 2 denier to about 3.5 denier.

6. The composition of claim 1, where the fibers are silk fibers, preferably having a length of about 1 μm to about 10 μm.

7. The composition of claim 1, wherein the composition comprises the at least one latex film-forming agent having a low glass transition temperature (Tg) in an amount of from about 40% to about 67% by weight with respect to the total weight of the composition, preferably from about 50% to about 55% by weight with respect to the total weight of the composition.

8. The composition of claim 1, wherein the composition comprises the at least one latex film-forming agent having a low glass transition temperature (Tg) in an amount of from about 10% to about 30% by weight with respect to the total weight of the composition, preferably from about 10% to about 20% by weight with respect to the total weight of the composition.

9. The composition of claim 1, further comprising at least one ingredient selected from the group consisting of waxes, surfactants, oils, and mixtures thereof.

10. The composition of claim 1, wherein the composition is devoid of waxes, surfactants, and oils.

11. The composition of claim 1, further comprising at least one coloring agent.

12. The composition of claim 1, wherein the composition is devoid of coloring agent.

13. A method of applying artificial eyelashes and/or eyebrows to natural eyelashes and/or eyebrows comprising applying the composition of claim 1 to natural eyelashes and/or eyebrows, applying artificial eyelashes and/or eyebrows to the natural eyelashes and/or eyebrows upon which the composition has been applied, and allowing the composition to dry so that the artificial eyelashes and/or eyebrows attach to the natural eyelashes and/or eyebrows.
